(19) United States
(12) Patent Application Publication
(10) Pub. No.: US 2023/0001122 A1
(43) Pub. Date: Jan. 5, 2023

(54) SELECTABLE AROMATHERAPY AUGMENTATION OF OXYGEN DELIVERY SYSTEM

(71) Applicant: BOA Nutrition, Inc., Raleigh, NC (US)

(72) Inventors: Hank Durschlag, Raleigh, NC (US); Jon Pritchett, Raleigh, NC (US)

(21) Appl. No.: 17/839,115

(22) Filed: Jun. 13, 2022

Related U.S. Application Data
(60) Provisional application No. 63/209,787, filed on Jun. 11, 2021.

(51) Int. Cl. A61M 16/06; A61M 16/20

(52) U.S. Cl. CPC A61M 16/06; A61M 16/20; A61M 2202/0208

(57) ABSTRACT

Disclosed are an apparatus, system, a kit and a method for delivering supplemental oxygen together with an aromatherapy active. The system comprises one or more oxygen containers for delivering of supplemental oxygen comprising between 35 and 40 percent oxygen—to a user. The system comprise one or more selectable aromatherapy pods, which may be incorporated into the supplemental oxygen delivery system to increase user satisfaction and promote the frequent use of the supplemental oxygen therapy to improve athletic performance.

### CROSS-REFERENCE TO RELATED APPLICATIONS

The present disclosure claims the priority benefit of U.S. provisional patent application 63/209,787 filed Jun. 11, 2021, the disclosure of which is incorporated by reference herein.

### BACKGROUND OF THE INVENTION

### 1. Field of the Disclosure

The present disclosure is generally related to the delivery of supplemental oxygen to improve athletic performance and specifically integrate aromatherapy into the delivery of supplemental oxygen.

### 2. Description of the Related Art

It has been demonstrated that 100% oxygen administration increased mental and physical performance, including decreased reaction times and improved fatigue recovery. The delivery of 100% oxygen is difficult without sealed masks, making it impractical during athletic events. Recent studies have shown that a relatively low percentage, 30-40% hyperoxic air may deliver a similar decrease in reaction time and increased cognitive performance. Therefore, it is desirable to have a system for the delivery of hyperoxic air to athletes in an easy-to-use fashion.

Studies have demonstrated aerobic fitness to be higher in users who integrated aromatherapy into exercise and other athletic activities. For example, cardamom has demonstrable effects on the autonomic nervous system as measured by a decrease in heart rate variability during exercise. Odors have been shown to have physiological effects on athletes during physical tasks such as conditioning, weight training, etc. For example, peppermint has been associated with subjects easing physical workload and temporal demand incurred during physical activity.

The effects on exercisers from aromatherapy have been shown to include reduced fatigue during exercise, improved feeling during exercise, and positively impact exercise satisfaction and persistence. Therefore, it would be desirable to have a system to integrate both supplemental oxygen and aromatherapy into an athlete's training regime as a mechanism to improve performance.

### SUMMARY OF THE CLAIMED INVENTION

Disclosed herein is an apparatus, a system, a kit and/or a method of delivering both aromatherapy and supplemental oxygen to a user. In one embodiment, the system of the present invention comprises an aromatherapy augmented supplemental oxygen delivery apparatus. And such apparatus comprises at least one oxygen container and, at least one aromatherapy delivery mechanism attached to the oxygen container, wherein a user may select at least one aromatherapy delivery mechanism attached to the pressurized oxygen container.

Another embodiment of the present invention relates to a kit, while yet another embodiment of the present invention relates to a method of delivering both oxygen and aromatherapy to a user. The method comprises one or more steps of setting the supplemental oxygen delivery apparatus, selecting a desired aromatherapy pod, allowing outlet 110 to receive and produce a mixture of the desired oxygen composition with the desired aromatherapy, and delivering such mixture to a user. The method may further comprise a step of allowing the valve 202 to receive a aromatherapy.

### BRIEF DESCRIPTIONS OF THE DRAWINGS

FIG. 1 illustrates a selectable aromatherapy augmentation of oxygen delivery system, according to an embodiment.

FIG. 2 illustrates an outlet, according to an embodiment.

### DETAILED DESCRIPTION

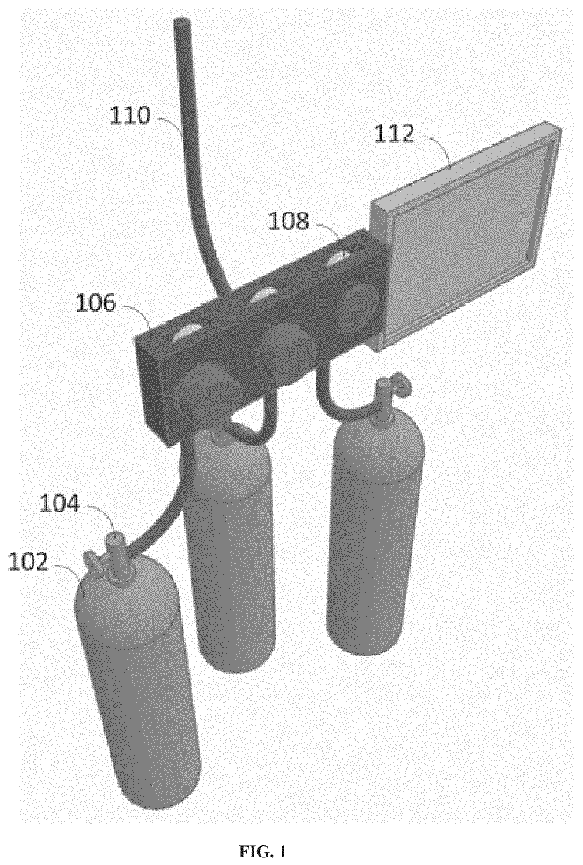

The present invention provides an apparatus, a system, a kit and a method of delivering and benefiting both aromatherapy and supplemental oxygen therapy. The apparatus or the system may be used in various occasions, such as for improving a user's daily life, benefiting a user's performance, and/or used in a therapy. Improving the user's daily life may include, but not limited to, reducing stress, improving alertness, enhancing sleep, and/or promoting emotional, physical, and cognitive well-being. The user's performance may include, but not limited to, performance in a sport, performance in a physical task, or a performance in a mental task, or any combinations thereof. The said therapy may include, but not limited to, a complementary therapy, a mind body therapy, a sensory art therapy, a phytotherapy, a psychotherapy, or any combinations thereof. The apparatus or the system may be the selectable aromatherapy oxygen augmentation system shown in FIG. 1. The apparatus or the system may comprise one or more oxygen containers 102. The oxygen containers may comprise tanks, canisters, other storage vessels suitable for holding a gas under pressure, or any other means or systems well known in the art for oxygen storage. Oxygen containers may comprise different sizes and/or shapes. As an example, the oxygen containers may range in size from small handheld bottles to large industrial use tanks. As another example, the oxygen containers may be in the shape of a canister, a tank, a vessel, or a pump. The system may comprise one or more regulators 104. The regulators may be attachable to the oxygen containers, or pre-attached to the oxygen container. The regulators may have one or more functions. The functions may comprise controlling the release of oxygen from the oxygen container 102. Such controlling may comprise controlling the pressure, the speed, and/or the oxygen composition coming out of the oxygen container 102. Regulators may be pressure-reducing devices designed to regulate the release of oxygen from the oxygen container 102 at a lower pressure than the user may safely use. Regulators may deliver a pulse of oxygen when the user inhales. In one embodiment, the regulator 104 may have a mechanical or electronic counter that may measure the number of breaths of oxygen taken, duration, oxygen uptake volume, or any other parameters related to the inhale cycle In one embodiment, the regulators may comprise the function of adjusting oxygen composition coming out of the oxygen containers. Such oxygen composition may benefit the user, such as to improve reaction time, to increase accuracy, and/or reduce recovery time. Such oxygen composition may comprise oxygen in the volume ratio (v/v) from about 100% to about 15%. In one embodiment, the oxygen composition may comprise oxygen of about 100%, about 95%, about 90%, about 80%, about 85%, about 75%, about 70%, about 65%, about 60%, about 55%, about 50%, about 48%, about 45%, about 43%, about 40%, about 38%, about 35%, about 32%, about 30%, about 28%, about 25%, about 23%, about 20%, about 15%, about 10%, and about 5%. In yet another embodiment, the oxygen amount delivered from the regulator 104 may comprise a range, such as in a range of about 100% to about 90%, about 100% to about 80%, about 90% to about 80%, about 80% to about 70%, about 70% to about 60%, about 60% to about 50%, about 50% to about 40%, about 55% to about 45%, about 40% to about 30%, about 45% to about 35%, about 30% to about 20%, about 35% to about 25%, about 20% to about 10%, or about 25% to about 15%. In one embodiment, the regulator 104 may be calibrated to deliver a an oxygen composition that comprises about 41% oxygen to improve reaction time, increase accuracy, and/or reduce recovery time.

The apparatus or the system may comprise at least one aromatherapy selector 106. The at least one aromatherapy selector may select at least one aromatherapy pod 108. The at least one aromatherapy pod 108 may house at least one active for an aromatherapy. The at least one active may comprise an essential oil, an active extracted from botanicals, and/or an essence isolated from botanicals. The aromatherapy selector may allow a user to select a specific aromatherapy pod to be combined with the oxygen composition from the regulator 104. And such a combination may be inhaled by a user through the outlet 110. In one embodiment, there may be multiple oxygen containers 102 connected through regulators 104 to a single aromatherapy selector 106 with a single outlet 110, and a user may select which aromatherapy pod 108 in the aromatherapy selector 106 is released with the oxygen composition through the outlet 110. In one embodiment, there may be an outlet 110 for each aromatherapy pod 108, which may allow the user to select their desired aromatherapy pod 108 based on the outlet they choose. In one embodiment, there may be a receiver on the regulator 104 or the outlet 110 into which a selected aromatherapy pod 108 may be inserted. Embodiments may include one or more aromatherapy pods 108, which may incorporate aroma into the oxygen composition released from the at least one regulator 104. In one embodiment, an aromatherapy with a known therapeutic value, such as a eucalyptus-scented aromatherapy pod 108 may be used to relieve congestion. Embodiments may include one or more outlets 110 that allow the user to inhale the oxygen composition from the regulator 104 with some portion of at least one additional aromatherapy pod 108. The outlets 110 may be any types known in the art. An outlet 110 may be made of a flexible material, a rigid material, or made of a flexible material in one portion and a rigid material in another portion. An outlet 110 may comprise a plastic, silicone, or rubber hose connected to a mask that covers at least the user's nose and/or mouth. Embodiments may comprises a display 112 that may indicate or display information of the system. Such information may include, but not limited to, the aromatherapy selected, data from the regulator 104, and the content or life remaining in the oxygen container 102 or in the aromatherapy pod 108. In one embodiment, display 112 may be integrated with the aromatherapy selector 106. In one embodiment, data from the regulator 104 and/or the aromatherapy selector 106 may be wirelessly communicated to a mobile device, which may act as the display 112. The data collected may be integrated with a hydration regimen as a larger athletic performance optimization system in one embodiment. The system may further comprise one or more sensors for aromatherapy and/or one or more sensors for oxygen use (not shown in FIG. 1). The system may further comprise an integrated electronics that comprises a power supply, a controller, a memory and/or software that would be used to read the sensors and run the software to drive the display.

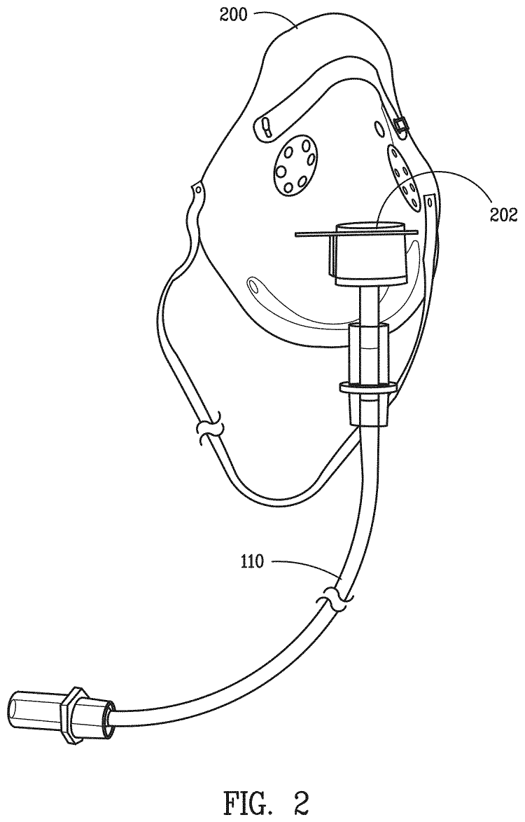

FIG. 2 illustrates an example of outlet 110. An outlet 110 may be flexible or rigid, or flexible in one portion and rigid in another portion. In one embodiment, the outlet 110 comprises a plastic, silicone, or rubber hose connected to a mask 200 that covers at least the user's nose and/or mouth. The mask 200 is detachable and replaceable from the system, In one embodiment, multiple types of masks 200 may be interchanged on the end of outlet 110 based on the application and the user's preferences. In one embodiment, multiple users may share the same outlet 110, but use interchanged masks 200, and/or an insert into the mask, for sanitary purposes. In one embodiment, an aromatherapy insert may be placed on the edge of the mask 200. The insert may be color-coded to indicate the type/flavor of the aromatherapy inserted. In one embodiment, the insert may have a permeable mesh that contains the contents of the aromatherapy pod 108 on the mesh to allow the user to experience the flavor and benefits of the aromatherapy. Masks 200 may have one-way valves 202 that allow air from the oxygen container 102 to reach the user while preventing air from being pushed into the outlet 110. In one embodiment, the one-way valve may serve as a receptacle for an aromatherapy pod 108. In one embodiment, the contents of the aromatherapy pod 108 may be integrated into outlet 110 directly. The content or life of the aromatherapy pod 108 may be visually indicated. For example, outlets 110 are color-coded to indicate the type/flavor of aromatherapy presented. Visual indicators may be a way to reinforce a user's preference for a given flavor/type of aromatherapy. This may encourage more frequent intake of supplemental oxygen, which has positively impacted athletic performance. Since delivering of supplemental oxygen is effectively integrated to an aromatherapy, it would bring enhanced even synergistic benefits of the two to an user. Additionally, the instant system is easy to use, yet have the flexibility of adjusting based on an user's individual needs. Such features would promote the frequent and habitual use of the system. For example, in American football, supplemental oxygen use is typically confined to exhausted players after a play or possession. Invigorating aromatherapy, such as eucalyptus or peppermint, used before the activity may allow the user to benefit from the improved response time that supplemental oxygen use has been demonstrated to deliver. Calming aromatherapy, such as lavender, may be used after the play or possession to relax the user. This connection of aromatherapy type/flavor with activity context may expand the context of supplemental oxygen use that becomes habitual. In one embodiment, the type/flavor of aromatherapy used in a given context may be chosen by a medical professional or athletic trainer systematically to optimize the athletic performance of a team.

The present invention further comprises a kit encompassing the aforementioned system. The kit may comprise the components illustrated in FIGS. 1-2. The kit may comprise the components explained above. The kit may comprise an instruction or an operation manual, which explain and guide user on operating the system, and/or how to trouble shooting the system by a user or a system management. Such an instruction or operation manual may be in a printed form, such as a guidance book or booklet, or may be displayable on the display 112.

The present invention further comprises a method of using the aforementioned system and/or the kit. Such a method may comprise one or more steps of setting the regulator 104 to a desired function, selecting a desired aromatherapy pod 108, allowing outlet 110 to receive and produce a mixture of the desired oxygen composition with the desired aromatherapy, and delivering such mixture to a user. The method may further comprise a step of allowing the valve 202 to receive a desired aromatherapy. The method may comprise allowing the display to provide data collected from any one ore more of the oxygen container, the regulator, the aromatherapy selector, the aromatherapy pod, or the valve. The method may comprise allowing the outlet 110 to display a color-code to indicate the type/flavor of the aromatherapy inserted.

The following examples are included to demonstrate some embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

For example, in American football, a supplemental oxygen may be desired for an exhausted player after a play or possession. Such supplemental oxygen may comprise oxygen in the range of 30-70% v/v in an oxygen composition, and such a composition may be delivered to the player through the instant system. In another example, an invigorating aromatherapy, such as with the active of eucalyptus or peppermint, may be used before the activity, and delivered to the user together with the supplemental oxygen, through the instant system. Such a delivery may improve user's response time. In yet another example, a calming aromatherapy, such as with lavender, may be used after a sport to relax the player. Using the instant system, such an aromatherapy may be easily combined with the delivery of supplemental oxygen to the user to achieve enhanced beneficial effects. The instant system is easy to use and facilitates frequent use. In one embodiment, the type/flavor of aromatherapy used in a given context may be chosen systematically by a medical professional or an athletic trainer to optimize the athletic performance of players in a team.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The terms “comprising,” “including” and “having” are used interchangeably in this disclosure. The terms “comprising,” “including” and “having” mean to include, but not necessarily be limited to the things so described. The terms “about” or “approximately,” as used in the application and claims, should be understood to include the recited values or a value that is three times greater or one third of the recited values.

What is claimed is:

1. An augmented aromatherapy delivery apparatus with supplemental oxygen, comprising:
a. at least one oxygen container;
b. at least one aromatherapy delivery mechanism attached to the oxygen container; and,
wherein a user may select at least one aromatherapy delivery mechanism attached to the oxygen container.

2. The augmented aromatherapy delivery apparatus of claim 1, wherein the at least one oxygen container is attached to the aromatherapy delivery mechanism through a regulator.

3. The augmented aromatherapy delivery apparatus of claim 1, wherein the at least one oxygen container comprises pressurized oxygen, or an oxygen composition.

4. The augmented aromatherapy delivery apparatus of claim 2, wherein the regulator comprises functions of adjusting the pressure or composition of oxygen released from the oxygen container.

5. The augmented aromatherapy delivery apparatus of claim 4, wherein the pressure or composition of oxygen released from the oxygen container is pre-determined by a medical professional, an athlete trainer, or an ordinary user.

6. The augmented aromatherapy delivery apparatus of claim 4, wherein the composition of oxygen released from the oxygen container comprises oxygen in the volume ratio (v/v) of about 80%, about 75%, about 70%, about 65%, about 60%, about 55%, about 50%, about 45%, about 40%, about 35%, about 30%, about 25%, about 20%, about 15%, or about 10%.

7. The augmented aromatherapy delivery apparatus of claim 4, wherein the oxygen released from the oxygen container comprises oxygen in the range of about 80% to about 70%, about 75% to about 65%, about 70% to about 60%, about 55% to about 45%, about 50% to about 40%, about 35%, about 25%, about 40% to about 30%, about 25% to about 15%, about 20% to about 10%.

8. The augmented aromatherapy delivery apparatus of claim 1, wherein the at least one aromatherapy delivery mechanism comprises at least one aromatherapy selector and at least one aromatherapy pods.

9. The augmented aromatherapy delivery apparatus of claim 1, wherein the at least one aromatherapy pod houses at least one aromatherapy active comprising an essential oil, an extract from botanicals, an essence isolated from botanicals.

10. The augmented aromatherapy delivery apparatus of claim 9, wherein the at least one aromatherapy active comprises eucalyptus, peppermint, lavender, or a combination thereof.

11. The augmented aromatherapy delivery apparatus of claim 1, further comprises an outlet, a mask, or a display.

12. The augmented aromatherapy delivery apparatus of claim 11, wherein the outlet combines oxygen released from the at least one oxygen container with the aromatherapy active released from the at least one aromatherapy delivery mechanism.

13. The augmented aromatherapy delivery apparatus of claim 11, wherein the mask is attachable to one end of the outlet.

14. The augmented aromatherapy delivery apparatus of claim 11, wherein the mask comprises a valve.

15. The augmented aromatherapy delivery apparatus of claim 11, wherein the valve is an one-way valve, only allowing oxygen released from the at least one oxygen container to reach the mask while preventing oxygen from being pushed into the outlet.

16. The augmented aromatherapy delivery apparatus of claim 11, wherein the valve serves as a receptacle for an aromatherapy active.

17. The augmented aromatherapy delivery apparatus of claim 11, wherein the display is integrated with the at least one aromatherapy delivery mechanism.

18. The augmented aromatherapy delivery apparatus of claim 11, wherein the display indicates the content level in the at least one oxygen container and the at least one aromatherapy pods.

19. A kit comprises:
(i) the augmented aromatherapy delivery apparatus of claim 1; and
(ii) an operation manual providing guidance on the use of the augmented aromatherapy delivery apparatus of claim 1.

20. A method of delivering both supplemental oxygen and an aromatherapy active to a user, comprising the steps of:
(i) setting at least one oxygen container to release supplemental oxygen;
(ii) selecting at least one aromatherapy pod to release an aromatherapy active;
(iii) allowing an outlet to receive the supplemental oxygen and the aromatherapy active; and
(iv) connecting the outlet to the user, thus delivering both the supplemental oxygen and the aromatherapy active to the user.
